(19) United States
(12) Patent Application Publication
(10) Pub. No.: US 2020/0058380 A1
(43) Pub. Date: Feb. 20, 2020

(54) PREDICTING THE PLACEBO RESPONSE AND PLACEBO RESPONDERS USING BASELINE PSYCHOMETRIC AND CLINICAL ASSESSMENT SCORE

(71) Applicant: THE REGENTS OF THE UNIVERSITY OF CALIFORNIA, OAKLAND, CA (US)

(72) Inventor: Ariana ANDERSON, MALIBU, CA (US)

(73) Assignee: THE REGENTS OF THE UNIVERSITY OF CALIFORNIA, OAKLAND, CA (US)

(21) Appl. No.: 16/321,774

(22) PCT Filed: Jul. 28, 2017
(86) PCT No.: PCT/US17/44483

§ 371 Date: Jan. 29, 2019

Related U.S. Application Data
(63) Continuation of application No. 62/368,558, filed on Jul. 29, 2016.

(51) Int. Cl. G16H 10/20; G06N 20/20; G16H 20/10

(52) U.S. Cl. CPC G16H 10/20; G16H 20/10; G06N 20/20

(57) ABSTRACT

The invention provides tools and methods for improving the quality and efficiency of clinical trials, including designing clinical trials that do not require a placebo group. In one aspect, the invention provides a method of identifying placebo responders. In another aspect, the invention provides a method for calculating and/or estimating a placebo risk score for one or more individual subjects. In yet another aspect, the invention provides a method for adjusting results of a clinical trial by removing the placebo responders and/or the portion of their response attributable to placebo effect. In an additional aspect, the invention provides a method of measuring the response of an individual to treatment that is not attributable to placebo effect.

This application claims the benefit of U.S. provisional application No. 62/368,558, filed Jul. 29, 2016, the entire contents of which are incorporated herein by reference.

### TECHNICAL FIELD OF THE INVENTION

The invention relates to tools, materials, and methods for improving the quality and efficiency of clinical trials, including methods for designing clinical trials, including clinical trials that can be performed without requiring use of a placebo, and for measuring the response of an individual to treatment that is not attributable to placebo effect. The invention also relates to methods for calculating and/or estimating a placebo risk score for one or more individual subjects. Such risk scores can be used to adjust the dosing of active medication.

### BACKGROUND OF THE INVENTION

The placebo effect is used to describe the effect of inert interventions to yield a positive treatment benefit. In studies of antipsychotic medications, the placebo response has been increasing since 1960, coinciding with a decrease in the estimated size of the medication response (Rutherford et al., 2014), with a significant interaction between the effective dose and publication year, baseline severity and trial duration. The placebo effect is greater in studies with a large number of trials, but was found to not be influenced by the frequency of clinician contact. The medication effect is larger in comparator studies than in placebo controlled studies, possibly because the patient realizes (s)he is guaranteed to receive an active medication.

### SUMMARY OF THE INVENTION

The invention provides tools and methods for improving the quality and efficiency of clinical trials, including methods for designing clinical trials. In one aspect, the invention provides a method of identifying placebo responders. This identification allows for removal of placebo responders before proceeding with a clinical trial, or adjusting the dosing of medication for placebo responders retained in the trial. In another aspect, the invention provides a method for calculating and/or estimating a placebo risk score for one or more individual subjects. Such risk scores can be used to adjust the dosing of active medication. In yet another aspect, the invention provides a method for adjusting results of a clinical trial by removing the placebo responders and/or the portion of their response attributable to placebo effect. In an additional aspect, the invention provides a method of measuring the response of an individual to treatment that is not attributable to placebo effect.

In one embodiment, the invention provides a method of identifying placebo responders in a treatment group, the method comprising measuring a plurality of symptoms in a first population of subjects receiving a placebo treatment before and after said treatment to obtain a plurality of placebo difference scores; performing a clustering analysis to create two distinct profiles of placebo difference scores measured, wherein a responder profile exhibits greater difference scores than a non-responder profile; and identifying subjects in the first population who exhibit the responder profile as placebo responders. In one embodiment, the method further comprises measuring the plurality of symptoms in a second population of subjects before receiving a psychopharmacologic treatment, and identifying subjects whose expected treatment difference scores will likely exhibit the responder profile of placebo responders. In one embodiment, the clustering analysis is a spectral clustering analysis.

In one embodiment, the invention provides a method of producing a placebo responder profile from a treatment group of subjects, the method comprising:
(a) measuring a plurality of symptoms in a first population of subjects receiving a placebo treatment before and after said treatment to obtain a plurality of baseline symptom scores and post-treatment symptom scores;
(b) generating a plurality of placebo difference scores, wherein a placebo difference score is the difference between the post-treatment symptom score of a subject and the baseline symptom score of the same subject;
(c) performing a clustering analysis to create two distinct profiles of placebo difference scores generated in (b), wherein a profile associated with placebo responders exhibits greater total difference scores than a profile associated with non-responders; and
(d) analyzing the baseline symptom scores of the placebo responders identified in (c) to create a baseline placebo responder profile.

In one embodiment, the clustering analysis is a spectral clustering analysis. Optionally, the method further comprises:
(e) measuring the plurality of symptoms in a second population of subjects receiving a psychopharmacologic treatment before said psychopharmacologic treatment to obtain a plurality of baseline symptom scores; and
(f) identifying subjects in the second population whose baseline symptom scores exhibit the baseline placebo responder profile of (d) as placebo responders.

Additionally, the invention provides a method of obtaining a placebo quantified response score (PQRS) from a treatment group of subjects. In one embodiment, the method comprises:
(a) measuring a plurality of symptoms in a first population of subjects receiving a placebo treatment before and after said treatment to obtain a plurality of baseline symptom scores and post-treatment symptom scores;
(b) generating a plurality of placebo difference scores, wherein a placebo difference score is the difference between the post-treatment symptom score of a subject and the baseline symptom score of the same subject;
(c) modeling a total post-treatment symptom score measured after treatment with placebo as a function of baseline symptom scores; and
(d) generating a PQRS using the modeling of step (c) to predict a post-treatment symptom score attributable to placebo based on a baseline symptom score.

The method optionally further comprises:
(e) measuring the plurality of symptoms in a second population of subjects receiving a psychopharmacologic treatment before and after said psychopharmacologic treatment to obtain a plurality of baseline symptom scores and post-treatment symptom scores;
(f) generating a PQRS for each subject in the second population; and
(g) adjusting the post-treatment scores measured in the second population of subjects using the PQRS for that subject.

Also provided is a method of improving the quality and efficiency of a clinical trial. In one embodiment, the method comprises obtaining measured responses of a plurality of symptoms in a population of subjects prior to a trial treatment; analyzing the measured responses obtained to generate a placebo quantified response score (PQRS); measuring the subjects' responses to treatment with either a trial medication or a placebo; and adjusting the measured responses obtained in (c) to account for the PQRS. In one embodiment, the adjusting comprises removing measured responses of subjects whose PQRS identifies them as placebo responders from the responses measured in the preceding step, and/or subtracting the PQRS from the measured response of each subject. In some embodiments, the latter two steps are performed only for those subjects whose PQRS identifies them as placebo non-responders.

The plurality of symptoms, in one representative example, is the set of symptoms of the Positive and Negative Syndrome Scale (PANSS). In another representative example, the plurality of symptoms is the set of symptoms of the Hamilton Depression Rating Scale (HAM-D). In another representative example, the plurality of symptoms is the set of symptoms of the Hamilton Anxiety Rating Scale (HAM-A). In another representative example, the plurality of symptoms is the set of symptoms in the Beck's Depression Inventory, the Montgomery-Åsberg Depression Rating Scale (MADRS), or the Hospital Anxiety and Depression Scale (HADS). In some embodiments, the PQRS identifies a subject as a placebo responder if a designated threshold is met, wherein the threshold distinguishes placebo responders from placebo non-responders using the methods described herein. In one embodiment, the PQRS is determined by modeling as described herein, for example, by modeling the total change in scores for placebo-treated groups using the baseline symptom scores, and using this model to predict for new patients the PQRS. In a typical embodiment, the trial medication is a psychopharmacologic treatment. Examples of the psychopharmacologic treatment or trial medication include, but are not limited to, one or more medications selected from the group consisting of Olanzapine, Paliperidone, Paliperidone Palmitate, Quetiapine, and Risperidone.

The methods described herein, in some embodiments, comprise obtaining measured responses that comprise functional magnetic resonance imaging (fMRI) of the brain of the subjects. Measurement of fMRI can be used to measure the placebo response as well as the treatment response (Anderson and Cohen, 2013, Stud. Health Technol. Inform. 184:6-12).

Additionally provided is a method of performing clinical trial of a psychopharmaco-logic treatment. In one embodiment, the method comprises obtaining measures of a plurality of symptoms in a population of subjects prior to a trial treatment; analyzing the measures obtained to generate a placebo quantified response score (PQRS); measuring the subjects' responses to treatment with either a trial medication; and adjusting the measures obtained in the preceding step to account for the PQRS. The clinical trial does not require administration of a placebo. The PQRS model used in this embodiment has been trained using pre- and post-treatment data in a placebo-treated group from a prior study. Additionally, a PQRS can be created using two active medications. In the two-medication embodiment, it would be assumed that the medications were mechanistically different, and their “overlap” was the placebo. This may not necessarily be a true placebo measure, depending on the medications. For example, SSRIs would act similarly to each other.

Also provided is a method of adjusting dosing of active medication within a clinical trial based upon a patients' PQRS and anticipated placebo responder status, whereby patients with a high PQRS and/or anticipated placebo responders are assigned a lower dose of active medication.

The invention additionally provides a device, such as a computer or other programmable instrument, which has been programmed to perform a method described herein. In one embodiment, the device is disposed to receive data regarding symptom scores obtained from subjects before and after treatment with placebo. The device is further disposed to perform analysis of the data and modeling to identify a placebo responder profile, to distinguish between placebo responders and non-responders, and/or to generate a PQRS. The device can also be used to predict a subject's response to medication using a baseline assessment score.

In one embodiment, the invention provides a system for performing a clinical trial. In a representative example, the system comprises:
(a) a computer readable memory storing data describing measured responses of a plurality of symptoms in a population of subjects prior to a trial treatment;
(b) an analyzer that generates a placebo quantified response score (PQRS) from measured responses stored as the data of (a);
(c) an analyzer that receives the subjects' measured responses to treatment with a trial medication; and
(d) a processor that adjusts the measured responses obtained in (c) to account for the PQRS.

In another representative example, the system comprises:
(a) means for obtaining measured responses of a plurality of symptoms in a population of subjects prior to a trial treatment;
(b) means for analyzing the measured responses obtained in (a) to generate a placebo quantified response score (PQRS);
(c) means for measuring the subjects' responses to treatment with a trial medication; and
(d) means for adjusting the measured responses obtained in (c) to account for the PQRS;
   wherein the clinical trial does not require administration of a placebo.

In another embodiment, the invention provides a computer system for analyzing data resulting from a clinical trial. In one embodiment, the system comprises:
(a) a first processor that analyzes measured responses of a plurality of symptoms in a population of subjects prior to a trial treatment to generate a placebo quantified response score (PQRS);
(b) a second processor that analyzes the subjects' measured responses to treatment with a trial medication; and
(c) a third processor that adjusts the measured responses obtained in (b) to account for the PQRS, wherein the first, second, and third processors are the same processor, separate processors, or a combination thereof.

Also provided is a computer readable non-transitory storage medium storing software for analyzing clinical data. In one embodiment, the software comprises:

(a) software that analyzes measured responses of a plurality of symptoms in a population of subjects prior to a trial treatment to generate a placebo quantified response score (PQRS):

(b) software that analyzes the subjects' measured responses to treatment with a trial medication; and

(c) software that adjusts the measured responses obtained in (b) to account for the PQRS.

### BRIEF DESCRIPTION OF THE DRAWINGS

FIG. 1. Schematic depiction of change patterns of placebo responders, identified by spectral clustering within placebo group. Placebo responders had a larger relative score change in the negative symptom domain than placebo non-responders.

FIG. 2. Schematic depiction of change patterns of placebo non-responders, identified by spectral clustering within the placebo group. Placebo Non-responders worsened in all symptom domains except for Anxiety/Depression, where they actually improved slightly.

FIG. 3. Placebo assigned patients were divided two groups based upon patterns in their change scores across the placebo intervention, under the hypothesis that the pattern of the score, and not just the magnitude, is significant in identifying placebo responders. These patterns of change were associated with overall change; placebo responders had higher baseline PANSS scores (were more severely ill). 1=Non-responder, 2=Responder.

FIG. 4. Within the medicated patient group, patients classified as placebo responders also were more severely ill than placebo non-responders. The placebo responders and non-responders though had less distinguishable total change scores, suggesting an interaction between the medication and the placebo response. 1=Non-responder, 2=Responder.

FIG. 5. Within the medication group, “placebo responder” patients had a higher predicted placebo effect than predicted “non-responders”. This increased for larger baseline PANSS scores.

FIG. 6. Stronger medication effects were associated with a decreased placebo effect. Medication effects were estimated using the difference between the observed treatment effect and the estimated placebo effect, within patients who had received an active drug.

FIG. 7. Flow chart depicting representative method steps to improve the design and/or quality of a clinical trial. As represented in the box at the left 702, at the outset, one can perform a baseline assessment (measuring a plurality of symptoms, for example) that is used to predict placebo responders 706 and/or to calculate a placebo quantified response score (PQRS). Optionally, the subjects identified as predicted placebo responders are removed from the study 710. Those subjects predicted to be placebo non-responders 704 are then randomized 708 for assignment to either the placebo group 712 or the active treatment group 714. The PQRS can then be used to adjust the final measures 716, 718, taking into account the placebo effect for each individual subject.

FIG. 8. Flow chart depicting representative method steps to identify and remove placebo responders using baseline symptom scores.

FIG. 9. Flow chart depicting representative method steps to adjust trial responses for the placebo quantified response score (PQRS).

FIG. 10. Flow chart depicting representative method steps for PQRS Trial Design, predicting placebo responders.

FIGS. 11A-11B. Distribution of Change Scores, assessed using the HAM-D 17 over an 8-week time period.

FIGS. 12A-12B. Following clustering, the “placebo responder” subgroup was identified by the total change in HAM-D scores (12B). This also corresponds to the patients with the greatest baseline PANSS scores (12A).

FIG. 13. The Placebo Responder group was best predicted by baseline symptoms of the HAM-D, age, and BMI. Although treatment assignment was also used to predict the placebo responder group, this variable had low variable importance, suggesting that the intervention did not interact with the placebo responder subtypes.

FIG. 14. The most important predictor in a random forest's model of a subjects total treatment response was the predicted placebo responder status. Other important predictors included pain, BMI, Age, and various symptoms from the other assessment instruments used.

FIG. 15. The most important predictor in an rpart decision tree model of a subject's total score change was whether the subject was a predicted placebo responder. Subjects who were in group 2 (placebo non-responders) had a lower total HAM-D score change, largely dependent upon their baseline symptoms. Subjects who were placebo responders had a score change which was dependent upon their pain level and BMI.

FIGS. 16A-16B. The predictors of total treatment response were different for the placebo and active-treatment groups.

FIG. 17. The total treatment response was predicted using baseline pain and intervention, including an interaction effect for these covariates. The interaction effect was not significant in a chi-square test comparing pain and depression treatment response. Increasing levels of pain were negatively associated with treatment response (p<0.10) in the baseline model.

FIG. 18. The strongest predictor of the PQRS was the total baseline HAM-D scores of a subject.

FIGS. 19A-19B. The drug effect is the score change difference seen between the placebo and compound. When the treatment response is compared between the compound and placebo groups for baseline HAM-D scores (FIG. 19A), the groups are separable for a narrow score range where the compound is superior to the placebo. When incorporating the PQRS (FIG. 19B), the treatments are separable for a broader range of scores. Shaded areas indicate 95% confidence intervals.

### DETAILED DESCRIPTION OF THE INVENTION

The invention is based on the unexpected discovery that, using baseline PANSS scores (and other baseline scores), it is possible to predict at baseline both who will respond to a placebo treatment, and how strongly they will respond. Identifying placebo responders at onset allows those subjects to be removed from the study, thus allowing the medication effect to be estimated only within subjects for whom it is most likely to benefit. Including the estimated placebo response within the trial analyses allows the medication effect to be estimated on a per-subject basis, above and beyond his unique placebo response.

The invention described herein thus provides a new trial design, one where (1) placebo responders are removed prospectively, without the need for a placebo lead-in, and (2) treatment responses are analyzed after holding constant for the likely placebo response in that individual patient-simulating a cross-over study without the second placebo phase actually being performed. Moreover, the PQRS allows estimation of the medication effect after accounting for the effect of the medication. Finally, in comparator trials, adjusting for the expected placebo response using the PQRS would allow medications to be compared directly, while subtracting out the unique placebo-related changes within that subject. These benefits—identifying placebo responders and estimating the placebo response in patients receiving a medication—are all performed without actually administering a placebo intervention. This, in turn, leads to a shortened study, as well as reducing costs.

### Definitions

All scientific and technical terms used in this application have meanings commonly used in the art unless otherwise specified. As used in this application, the following words or phrases have the meanings specified.

As used herein, a “profile of difference scores” refers to a set of difference scores for a set of symptoms, some of which may be positive and some of which may be negative. Two profiles of difference scores are distinct if the degree or direction (positive or negative) of difference scores are sufficiently different across a plurality of measures (e.g., a plurality of symptoms) that a statistically significant clustering, or lack of identity between the two profiles, is determined by cluster analysis.

As used herein, “measured responses” or “measures” refers to responses obtained when evaluating a patient for symptoms. These measures can be obtained both before and after treatment.

As used herein, a “greater difference score” refers, in the context of response to treatment with placebo, a greater overall reduction or improvement in symptoms.

As used herein, a “processor” refers to a device capable of processing information, such as in the form signal processing. One example of a processor is a digital signal processor circuit or an application specific integrated circuit (ASIC). One or more processors may be contained within a single device.

As used herein, an “analyzer” refers to a device capable of analyzing data. One or more analyzers may be contained within a single device.

As used herein, “a” or “an” means at least one, unless clearly indicated otherwise,

### Methods

The invention provides tools and methods for improving the quality and efficiency of clinical trials, including methods for designing clinical trials. In one aspect, the invention provides a method of identifying placebo responders. This identification allows for removal of placebo responders before proceeding with a clinical trial, or adjusting the dosing of medication for placebo responders retained in the trial. In another aspect, the invention provides a method for calculating and/or estimating a placebo risk score for one or more individual subjects. Such risk scores can be used to adjust the dosing of active medication. In yet another aspect, the invention provides a method for adjusting results of a clinical trial by removing the placebo responders and/or the portion of their response attributable to placebo effect. In an additional aspect, the invention provides a method of measuring the response of an individual to treatment that is not attributable to placebo effect.

In one embodiment, the invention provides a method of identifying placebo responders in a treatment group, the method comprising measuring a plurality of symptoms in a first population of subjects receiving a placebo treatment before and after said treatment to obtain a plurality of placebo difference scores; performing a clustering analysis to create two distinct profiles of placebo difference scores measured, wherein a responder profile exhibits greater difference scores than a non-responder profile; and identifying subjects in the first population who exhibit the responder profile as placebo responders. In one embodiment, the clustering analysis is a spectral clustering analysis. In one embodiment, the method further comprises measuring the plurality of symptoms in a second population of subjects before receiving a psychopharmacologic treatment, and identifying subjects whose expected treatment difference scores will likely exhibit the responder profile of placebo responders.

In one embodiment, the invention provides a method of producing a placebo responder profile from a treatment group of subjects, the method comprising:
(a) measuring a plurality of symptoms in a first population of subjects receiving a placebo treatment before and after said treatment to obtain a plurality of baseline symptom scores and post-treatment symptom scores;
(b) generating a plurality of placebo difference scores, wherein a placebo difference score is the difference between the post-treatment symptom score of a subject and the baseline symptom score of the same subject;
(c) performing a clustering analysis to create two distinct profiles of placebo difference scores generated in (b), wherein a profile associated with placebo responders exhibits greater total difference scores than a profile associated with non-responders; and
(d) analyzing the baseline symptom scores of the placebo responders identified in (c) to create a baseline placebo responder profile.

In one embodiment, the clustering analysis is a spectral clustering analysis. Optionally, the method further comprises:
(e) measuring the plurality of symptoms in a second population of subjects receiving a psychopharmacologic treatment before said psychopharmacologic treatment to obtain a plurality of baseline symptom scores; and
(f) identifying subjects in the second population whose baseline symptom scores exhibit the baseline placebo responder profile of (d) as placebo responders.

Also provided is a method of improving the quality and efficiency of a clinical trial. In one embodiment, the method comprises obtaining measured responses of a plurality of symptoms in a population of subjects prior to a trial treatment; analyzing the measured responses obtained to generate a placebo quantified response score (PQRS); measuring the subjects' responses to treatment with either a trial medication or a placebo; and adjusting the measured responses obtained in (c) to account for the PQRS. In one embodiment, the adjusting comprises removing measured responses of subjects whose PQRS identifies them as placebo responders from the responses measured in the preceding step, and/or subtracting the PQRS from the measured response of each subject. In some embodiments, the latter two steps are performed only for those subjects whose PQRS identifies them as placebo non-responders.

The plurality of symptoms, in one representative example, is the set of symptoms of the Positive and Negative Syndrome Scale (PANSS). In another representative example, the plurality of symptoms is the set of symptoms of the Hamilton Depression Rating Scale (HAM-D). In another representative example, the plurality of symptoms is the set of symptoms of the Hamilton Anxiety Rating Scale (HAM-A). In another representative example, the plurality of symptoms is the set of symptoms in the Hospital Anxiety and Depression Scale (HADS). In some embodiments, the PQRS identifies a subject as a placebo responder if a designated threshold is met, wherein the threshold distinguishes placebo responders from placebo non-responders using the methods described herein. In one embodiment, the PQRS is determined by modeling as described herein, for example, by modeling the total change in scores for placebo-treated groups using the baseline symptom scores, and using this model to predict for new patients the PQRS. In a typical embodiment, the trial medication is a psychopharmacologic treatment. Examples of the psychopharmacologic treatment or trial medication include, but are not limited to, one or more medications selected from the group consisting of Olanzapine, Paliperidone, Paliperidone Palmitate, Quetiapine, and Risperidone.

The methods described herein, in some embodiments, comprise obtaining measured responses that comprise functional magnetic resonance imaging (fMRI) of the brain of the subjects. Measurement of fMRI can be used to measure the placebo response as well as the treatment response (Anderson and Cohen, 2013, Stud. Health Technol, Inform. 184:6-12).

Additionally provided is a method of performing clinical trial of a psychopharmacologic treatment. In one embodiment, the method comprises obtaining measures of a plurality of symptoms in a population of subjects prior to a trial treatment; analyzing the measures obtained to generate a placebo quantified response score (PQRS); measuring the subjects' responses to treatment with either a trial medication; and adjusting the measures obtained in the preceding step to account for the PQRS. The clinical trial does not require administration of a placebo. The PQRS model used in this embodiment has been trained using pre- and post-treatment data in a placebo-treated group from a prior study.

Also provided is a method of adjusting dosing of active medication within a clinical trial based upon a patients' PQRS and anticipated placebo responder status, whereby patients with a high PQRS and/or anticipated placebo responders are assigned a lower dose of active medication.

### Computers, Software, Systems, and Related Media

The invention additionally provides a device, such as a computer or other programmable instrument, which has been programmed to perform a method described herein. In one embodiment, the device is disposed to receive data regarding symptom scores obtained from subjects before and after treatment with placebo. The device is further disposed to perform analysis of the data and modeling to identify a placebo responder profile, to distinguish between placebo responders and non-responders, and/or to generate a PQRS. The device can also be used to predict a subject's response to medication using a baseline assessment score.

In one embodiment, the invention provides a system for performing a clinical trial. In a representative example, the system comprises:
(a) a computer readable memory storing data describing measured responses of a plurality of symptoms in a population of subjects prior to a trial treatment;
(b) an analyzer that generates a placebo quantified response score (PQRS) from measured responses stored as the data of (a);
(c) an analyzer that receives the subjects' measured responses to treatment with a trial medication; and
(d) a processor that adjusts the measured responses obtained in (c) to account for the PQRS.

In another representative example, the system comprises:
(a) means for obtaining measured responses of a plurality of symptoms in a population of subjects prior to a trial treatment;
(b) means for analyzing the measured responses obtained in (a) to generate a placebo quantified response score (PQRS);
(c) means for measuring the subjects' responses to treatment with a trial medication; and
(d) means for adjusting the measured responses obtained in (c) to account for the PQRS;
   wherein the clinical trial does not require administration of a placebo.

In another embodiment, the invention provides a computer system for analyzing data resulting from a clinical trial. In one embodiment, the system comprises:
(a) a first processor that analyzes measured responses of a plurality of symptoms in a population of subjects prior to a trial treatment to generate a placebo quantified response score (PQRS),
(b) a second processor that analyzes the subjects' measured responses to treatment with a trial medication; and
(c) a third processor that adjusts the measured responses obtained in (b) to account for the PQRS,
   wherein the first; second, and third processors are the same processor, separate processors, or a combination thereof.

Also provided is a computer readable non-transitory storage medium storing software for analyzing clinical data. In one embodiment, the software comprises:
(a) software that analyzes measured responses of a plurality of symptoms in a population of subjects prior to a trial treatment to generate a placebo quantified response score (PQRS),
(b) software that analyzes the subjects' measured responses to treatment with a trial medication; and
(c) software that adjusts the measured responses obtained in (b) to account for the PQRS.

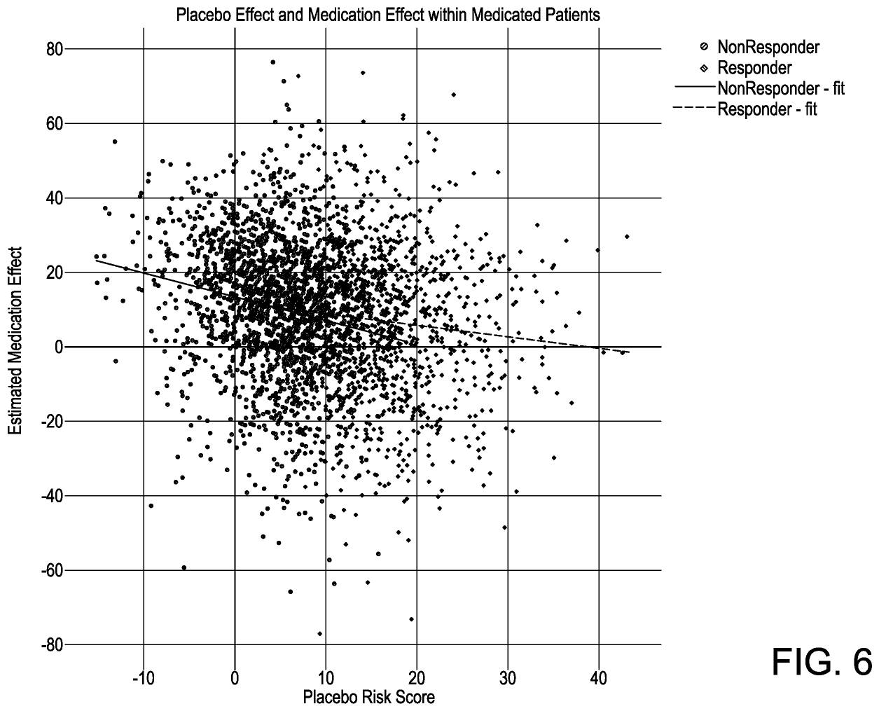

Likewise, the invention provides an article of manufacture that stores software in a non-transitory computer readable medium. The software is configured to direct one or more processors to perform the steps of one or more methods described herein, including, for example, method steps described in FIGS. 6-8.

### EXAMPLES

The following examples are presented to illustrate the present invention and to assist one of ordinary skill in making and using the same. The examples are not intended in any way to otherwise limit the scope of the invention.

### Example 1: Placebo-Quantified Response Scores for Prospective Placebo Responder Identification and Retrospective Medication Effect Estimation: A New Trial Design for CNS Drug Development

The placebo response is one of the greatest challenges in CNS drug development, and may operate more strongly in subjects receiving a placebo treatment. Placebo controlled trials then may put effective medications at a disadvantage, since the placebo response has a head start within the placebo group. Presented here is a novel trial design that prospectively predicts both placebo responders and the expected placebo response (Placebo Quantified Response Score [PQRS]) in patients assigned to both medication and placebo groups. Prospectively predicting placebo responders and the placebo response significantly increases the ability to predict the total medication response (p<0.001). Within patients receiving an active medication, a greater PQRS is associated with a larger total treatment response (p<0.001), and increases the ability to predict the total treatment response above and beyond the explanatory power of baseline PANSS, gender, age, and the medication received (p<0.001). Similar predictive ability was seen when identifying placebo responders (p<0.001). Removing placebo responders prospectively and holding constant the PQRS within medicated and unmedicated patients allows the placebo response to be corrected for and the true medication response to be estimated on a per-subject basis. Collectively, this trial design corrects for the inherent disadvantage of the placebo response within randomized controlled trials, and allows for a subject's medication response to be estimated above and beyond their unique placebo response.

The placebo response is used to describe the effect of inert interventions to yield a positive treatment benefit. In studies of antipsychotic medications, the placebo response has been increasing since 1960, coinciding with a decrease in the estimated size of the medication response (Rutherford et al., 2014) The placebo response is as strong as the medication response (Howick et al., 2013), but the total treatment effect is not merely the sum of these parts. In individuals with a higher placebo response, the medication response is diminished with a “ceiling” limiting the overall benefit of a medication (Lund et al., 2014). Cumulatively, the powerful placebo is able to subdue the ability to properly measure the effectiveness of active medications, since it is disproportionately stronger within the placebo group.

Many factors have been found to govern the magnitude of the placebo response. There was a significant interaction between the effective dose and publication year (Kemp et al., 2010), baseline severity and trial duration (Rutherford et al., 2014). The placebo effect is greater in studies with a large number of trials, but was found to not be influenced by the frequency of clinician contact [REF]. The medication effect is larger in comparator studies than in placebo controlled studies, possibly because the patient realizes the guarantee of an active medication [REF].

The increasing magnitude of the placebo response is a contributing factor to the 15% increase in failed Phase 2 clinical trials within the past decade (Kemp et al., 2010). This has led to an increased interest in identifying placebo responders for trial designs, such as the well-established placebo lead-in period where every subject is given a placebo at baseline, and the placebo responders can be removed from the study. More recently in studies of antidepressants, the efficacy of a placebo lead-in approach has been questioned (Faries et al., 2001). Placebo responders in the lead-in phase may be qualitatively different than the post-randomization placebo responders (Rabkin et al., 1987). Placebo lead-in trials may not be the most effective technique for identifying likely placebo responders during the entire trial.

Others have built upon the placebo lead-in trial design using secondary blocking procedures such as the sequential parallel comparison design (SPCD) (Fava et al., 2003) and the two-way enriched design (TED) (Ivanova and Tamura, 2011). In the SPCD, the subjects are treated from the onset with either an active medication or a placebo, with the placebo non-responders randomized again either to a medication or a placebo in a second trial phase. There was a nine-times greater effect size when removing placebo responders in the ADAPT-A in a study of depression, using the SPCD trial design (Fava et al., 2012). Similarly, the TED removes and randomizes both placebo responders in the first phase, and medication non-responders in the first phase. Following removal of first-phase patients, the remaining patients are once again randomized into a treatment or placebo intervention.

Described herein is a novel trial design that estimates an individual's placebo response and identifies likely placebo responders prospectively using only baseline assessments. This allows 1.) screening and removal of placebo responders within patients assigned to both medication and a placebo intervention, without performing a placebo lead-in phase, 2.) estimating an individual's symptom change due to the actual medication after accounting for his unique placebo-related symptom changes, similar to performing a cross-over trial without a second stage, and 3.) comparing the effectiveness of two active medications after accounting for the likely symptom change due to the placebo response within medicated patients. Collectively, this suggests a novel trial design where the placebo response is adjusted for, without needing to actually administer a placebo intervention. This adjustment would lead to an increase in statistical power by mitigating the placebo-response, and would allow for shorter trials that modifications of the placebo lead-in design.

This proposed trial design is demonstrated using a collection of placebo-controlled drug trials in schizophrenia, where patients were assessed throughout the trial using the Positive and Negative Symptom Scale (PANSS). The Positive and Negative Symptom Scale (PANSS) was created to measure symptom assessment in Schizophrenia, and rates the presence of 30 different symptoms on a 1-7 ordinal scale. The total score of all 30 symptoms is used as an estimate of disease severity. The PANSS is the most widely used scale in clinical trials with psychotic disorders worldwide, making it an ideal tool to study the placebo response.

### Materials and Methods

The placebo group data consists of 679 Schizophrenia baseline patients who received a placebo treatment, spanning 9 studies. The treatment group data consisted of 2,968 patients who were treated in 11 different studies, receiving five different medications: (Olanzapine, Paliperidone, Paliperidone Palmitate, Quetiapine, and Risperidone). Written informed consent for all patients was obtained after the study procedure was fully explained. Further demographic information of all patients are provided in Table 1.

TABLE 1
Demographic information of study population.

| ClinicalTrials.gov | | | | | | Baseline |
|---|---|---|---|---|---|---|
| Identifier | TREATMENTS | N TX | N Placebo | Male (%) | Age (SD) | PANSS (SD) |
| NCT00397033 | paliperidone extended-release | 164 | 0 | 0.61 | 39.78 (9.8) | 92.1 (11.3) |
| NCT00334126 | paliperidone extended-release, quetiapine | 170 | 34 | 0.58 | 35.92 (10.76) | 105.2 (13.9) |
| NCT00085748 | paliperidone extended-release | 63 | 30 | 0.26 | 69.58 (4.56) | 92.9 (9.2) |
| NCT00078039 | paliperidone extended-release, olanzapine | 446 | 109 | 0.51 | 37.15 (10.86) | 93.6 (10.7) |
| NCT00077714 | paliperidone extended-release | 242 | 73 | 0.73 | 41.81 (10.56) | 93.9 (11.8) |
| NCT00083668 | paliperidone extended-release, olanzapine | 426 | 90 | 0.66 | 36.74 (10.54) | 92.8 (12.4) |
| NCT00210717 | paliperidone palmitate, risperidone | 576 | 0 | 0.59 | 40.7 (11.69) | 81.1 (13) |
| NCT00210548 | paliperidone palmitate | 139 | 98 | 0.67 | 39.07 (10.36) | 90.8 (12.1) |
| NCT00101634 | paliperidone palmitate | 283 | 89 | 0.63 | 40.01 (11.28) | 91 (11.9) |
| NCT00590577 | paliperidone palmitate | 333 | 104 | 0.66 | 39.42 (10.7) | 87 (11) |
| NCT00074477 | paliperidone palmitate | 126 | 52 | 0.66 | 38.98 (10.47) | 87.3 (11.7) |

### Predicting Placebo Responders

Under the hypothesis that patients are dichotomous (placebo responders, placebo non-responders), spectral clustering was performed on the change score patterns within the placebo-assigned (PA) patients, dividing them into groups based upon the patterns of change within individual items. The cluster with the centroid having the most improvement was labeled as the “placebo responder” group. The other cluster was labeled as “non-responder.” An SVM classification model next was trained to predict whether a patient was a placebo responder using the baseline PANSS scores within the PA group.

This trained model was next tested by predicting within the medication-assigned (MA) group whether a subject was a placebo responder, and evaluating whether the placebo responder status was significant in predicting the total treatment response to medication above and beyond the total baseline PANSS score, medication received, age and gender. These models were compared using a chi-square test with nested models.

### Predicting the Placebo Quantified Response Score

The placebo quantified response score (PQRS) was next predicted in MA and PA patients. First, an SUM regression model was trained to predict the total change in PANSS score within the placebo group using the 30 baseline PANSS observations. The leave-one-out cross-validated scores were used in subsequent analyses as the predicted PQRS in the PA group. The predicted values of the SVM model in the MA group

As further validation, the next test performed was whether the PQRS in the MA group helps to predict the overall treatment response in patients receiving a medication, above and beyond the cumulative power of the baseline scores, gender, age, and treatment using a general linear model. This model is compared with a similar model, which additionally includes the PQRS as a covariate, using a chi-square test for nested models.

### Results

Predicting the treatment effect using baseline covariates and predicted placebo identification status, within the medication-assigned patient group is summarized in Table 2. The baseline derived placebo responder identification significantly increased the ability to predict the treatment response, within the medication-assigned patient group.

TABLE 2
Predicting the treatment effect using baseline covariates and
predicted placebo identification status.

| Variable | Estimate | Std. Error | t value | pr (>[t]) |
|---|---|---|---|---|
| (Intercept) | −12.7437 | 3.0984 | −4.11 | 0 |
| Predicted Placebo | 3.7638 | 0.7454 | 5.05 | 0 |
| Identification | | | | |
| Baseline PANSS total | 0.3752 | 0.0285 | 13.18 | 0 |
| Trt:P | 1.3394 | 1.1711 | 1.14 | 0.2528 |
| Trt:PP | −5.2977 | 1.1759 | −4.51 | 0 |
| Trt:Q | 6.2711 | 2.301 | 2.73 | 0.0065 |
| Trt:RC | −2.2879 | 1.5388 | −1.49 | 0.1372 |
| Age | −0.068 | 0.029 | −2.34 | 0.0192 |
| Gender:Male | −0.0905 | 0.6985 | −0.13 | 0.8969 |

The placebo responder identification helped to predict total treatment response above and beyond the predictive power of baseline PANSS, age, gender, and medication received (Table 2, p<0.001, chi-square test for nested models). It was associated with a stronger treatment response (p<0.001).

A greater PQRS is associated with a larger treatment response within the MA group (p<0.001) as shown in Table 3. Within MA patients, the PQRS predicts treatment response above and beyond the explanatory power of baseline PANSS, gender, age, and the medication received (p<0.001), chi-square test for nested models. Categorical variables are assessed with respect to a female patient taking Risperidone. The baseline-derived PQRS significantly (p<0.001) increased the ability to predict the treatment response in the medication-assigned patient group.

TABLE 3
Predicted change in PANSS score including Placebo Response Score,
within the medication-assigned patient group.

| Variable | Estimate | Std. Error | t value | pr (>[t]) |
|---|---|---|---|---|
| (Intercept) | −12.74 | 3.10 | −4.11 | 0.00 |
| Placebo Risk Store | 3.76 | 0.75 | 5.05 | 0.00 |
| Baseline PANSS total | 0.38 | 0.03 | 13.18 | 0.00 |
| Trt:P | 1.34 | 1.17 | 1.14 | 0.25 |
| Trt:PP | −5.30 | 1.18 | −4.51 | 0.00 |
| Trt:Q | 6.27 | 2.30 | 2.73 | 0.01 |
| Trt:RC | −2.29 | 1.54 | −1.49 | 0.14 |
| Age | −0.07 | 0.03 | −2.34 | 0.02 |
| Gender:Male | −0.09 | 0.70 | −0.13 | 0.90 |

The placebo quantified response score and the predicted placebo responder IDs are correlated, and together are not both significant in the model as shown in Table 4. Categorical variables are assessed with respect to a female patient taking Risperidone. The PQRS was a stronger predictor of treatment response than the placebo responder identification (p<0.001). This result suggests that study design should not use these two predictions in parallel. An alternative would be to remove placebo responders during baseline screening, and adjusting for the predicted placebo response (PQRS) in all subjects after the intervention.

TABLE 4
The placebo quantified response score predicted treatment response
more strongly than the placebo responder identification, within the
medication-assigned patient group.

| Variable | Estimate | Std. Error | t value | pr (>[t]) |
|---|---|---|---|---|
| (Intercept) | −11.62 | 3.10 | −3.75 | 0.00 |
| Baseline Total PANSS | 0.35 | 0.03 | 12.11 | 0.00 |
| Trt:P | 1.19 | 1.17 | 1.02 | 0.31 |
| Trt:PP | −5.33 | 1.17 | −4.55 | 0.00 |
| Trt:Q | 4.70 | 2.32 | 2.03 | 0.04 |
| Trt:RC | −2.52 | 1.53 | −1.64 | 0.10 |
| Age | −0.07 | 0.03 | −2.30 | 0.02 |
| Gender:Male | −0.22 | 0.70 | −0.31 | 0.75 |
| Placebo Risk Score | 0.27 | 0.06 | 4.47 | 0.00 |
| Placebo Responder | 0.68 | 1.01 | 0.67 | 0.50 |
| Identification | | | | |

### DISCUSSION

The clustering algorithm separated placebo-assigned patients into two distinct patterns of PANSS change scores, identified as responders and placebo non-responders. For placebo responders, there was a 26.44 point improvement in PANSS score, while placebo non-responders had a 6.77 point worsening in symptoms. For all patients, the average placebo effect was 9.03 points PANSS change, with a variance of 20.6 points.

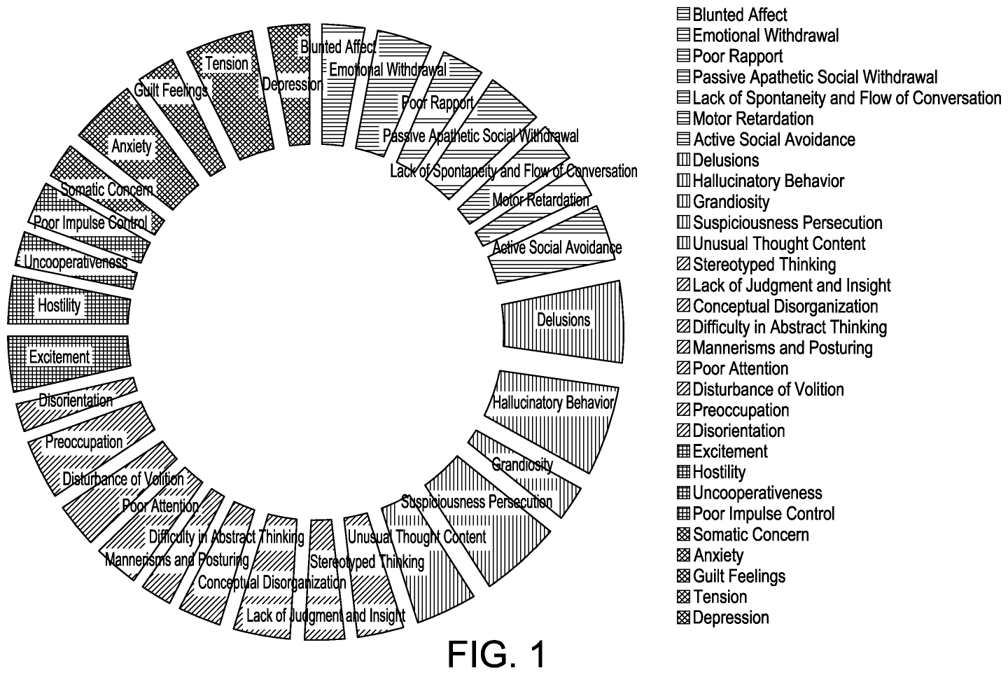

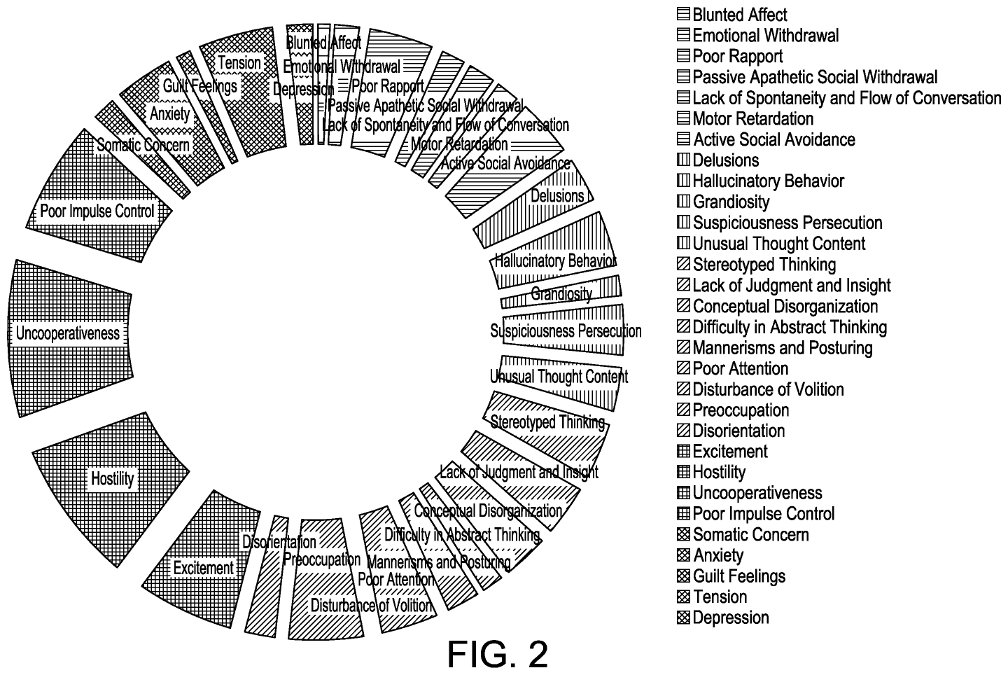

In FIG. 1 and FIG. 2, the change patterns are shown by factor domain for the placebo responders and non-responders. The placebo responders showed stronger magnitude of changes in all domains except for the anxiety/depression factors.

Within each factor domain, the placebo responders exhibited slightly different patterns. Placebo responders and non-responders all showed positive change (symptom decrease) in the positive, negative, excited, and disorganized domains. This change was stronger in the placebo responder group. Within the Anxiety/Depression domain, however, placebo responders showed markedly different patterns, with score increases for placebo responders, and score decreases for placebo non-responders.

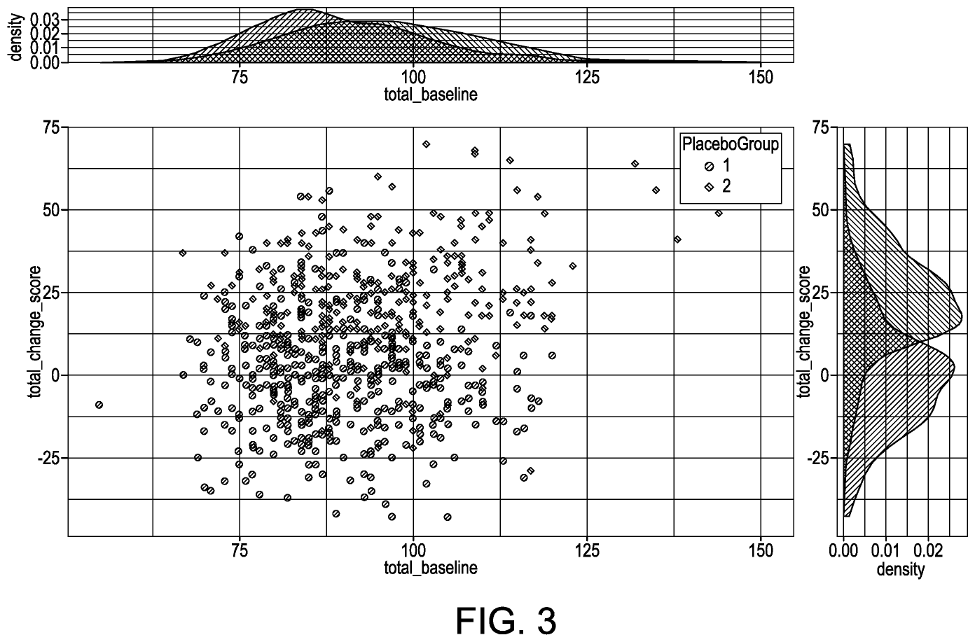

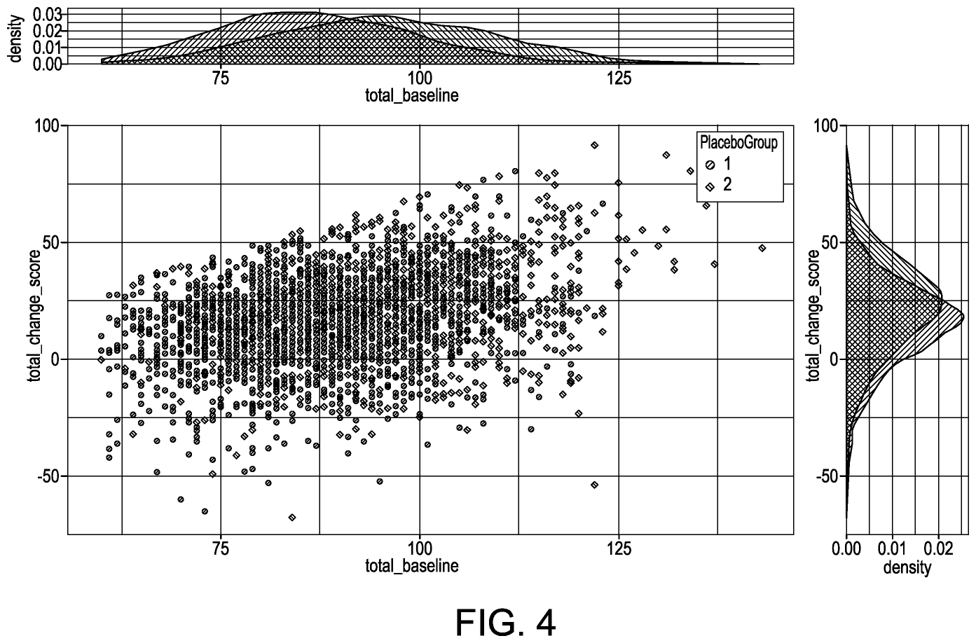

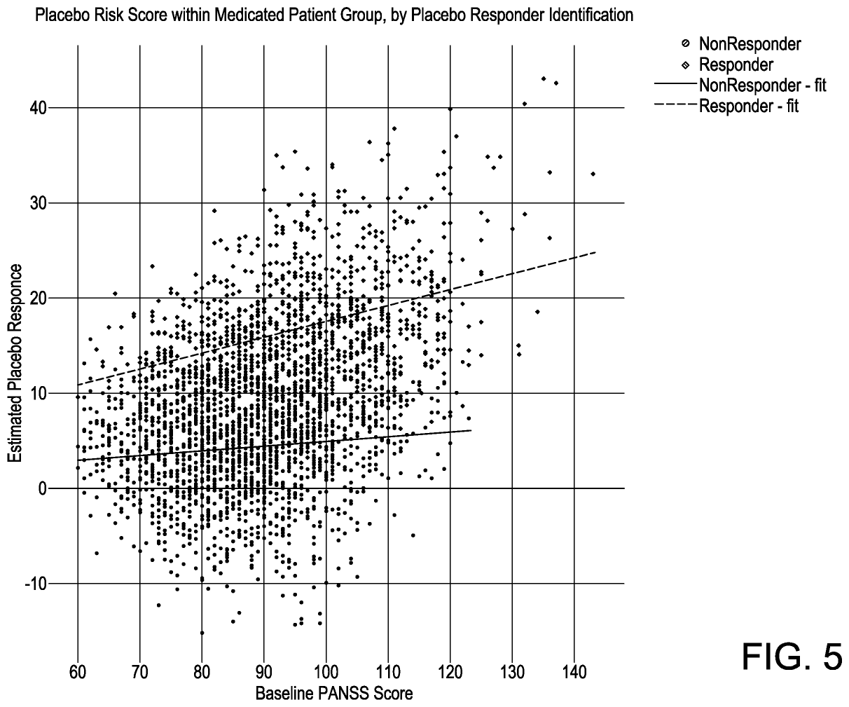

The relationship between the change scores and the baseline PANSS scores showed similar response patterns for placebo responders and non-responders, as shown in FIG. 3 and FIG. 4. Placebo responders had higher total change scores than placebo non-responders in the placebo group. Within the medication group, this pattern was still seen but less strong. This likely represents the blurring effect of the medication, which acts on both the placebo responders and non-responders alike.

TABLE 5
Total change in PANSS scores by placebo responder and non-
responder groups, within placebo patient group.

| | Placebo Non- | Placebo |
|---|---|---|
| Symptom Domain | Responder | Responder |
| Negative | −0.77 | 5.74 |
| Positive | −1.02 | 6.19 |
| Disorganized | −1.75 | 6.93 |
| Excited | −2.41 | 3.13 |
| Anxiety/Depression | −0.82 | 4.44 |

The relationship between the change scores and the baseline PANSS scores showed similar response patterns for placebo responders and non-responders, as shown in FIG. 3 and FIG. 4. Placebo responders had higher total change scores than placebo non-responders in the placebo group. Within the medication group, this pattern was still seen but the groups were less separable based upon the total treatment response. This suggests an interaction effect between the total treatment response and whether a subject is a likely placebo responder. This was verified in a secondary analysis, assessing whether the estimated pure medication effect (total treatment response-predicted placebo response) depended on whether a subject was a likely placebo responder. A higher PQRS was associated with a smaller pure medication effect.

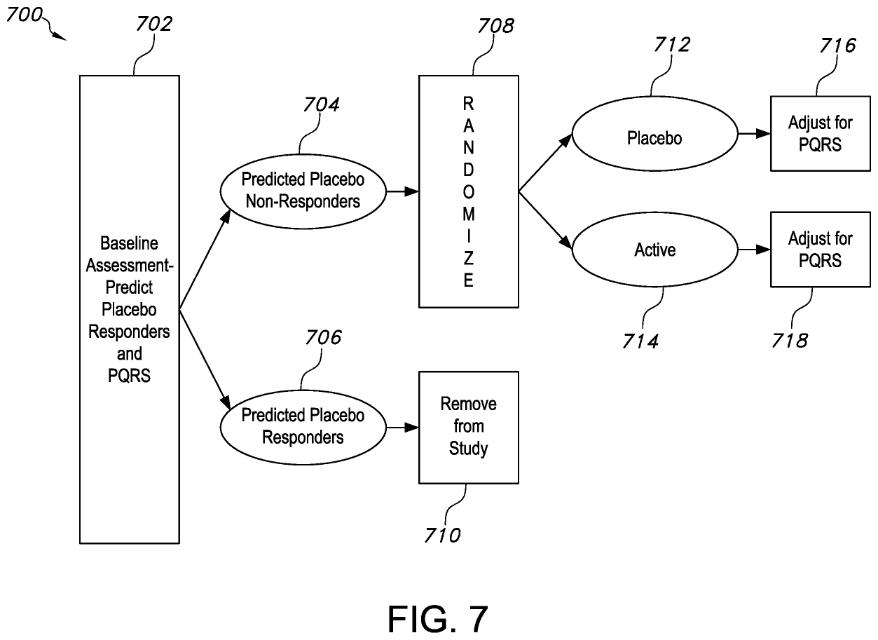

Finally, comparison was made, within MA patients, of the relationship between the PQRS and the estimated medication effect, where the medication effect was computed as the total change in PANSS score minus the estimated placebo effect (placebo response score), as shown in FIG. 7. Patients with stronger medication effects had smaller estimated placebo effects. This echoes a well-known hypothesis of a treatment “ceiling”, where the cumulative placebo effect and medication effect interact, and are bound by a common threshold which limits the total effect of the intervention.

Using baseline PANSS scores, it is possible to predict both who will respond to a placebo treatment, and how strongly they will respond. Identifying placebo responders at onset allows those subjects to be removed from the study, thus allowing the medication effect to be estimated only within subjects for whom it is most likely to benefit. Including the estimated placebo response within the trial analyses allows the medication effect to be estimated on a per-subject basis, above and beyond his unique placebo response.

These models were validated within the patients who had received medication, suggesting that the placebo patterns are both robust and replicable. Placebo responders showed stronger changes in the Positive, Negative, Excited and Disorganized domains, yet Placebo Non-responders showed the stronger changes in the Anxiety/Depression domains.

These models have implications for trial development, showing that it may be possible to predict how patients will react based upon their baseline PANSS patterns. The hypothesis is that including placebo quantified response score within the models will increase the ability to distinguish between the treatment and control conditions, by adjusting for the placebo effect directly within patients who were MA. This, in turn, will increase the statistical power and decrease the number of patients needed to establish statistical significance.

The tradeoff between the estimated medication effect and estimated placebo effect also suggests a “ceiling” to how strong the treatment response can be. This suggests that, within patients who are placebo non-responders, a higher dosage of medication may be necessary. More generally, this suggests that the typical study design, where the PA patient group is compared to the MA group directly, could be improved by stratification, a direction for future work, Prospectively predicting the subjects' expected placebo response would allow both for potential placebo responders to be screened before the trial, as well as the true medication effect within treated patients after accounting for that individual subject's placebo response, effectively separating the total treatment response into the separate parts (medication and treatment). Moreover, the same methods can be used to predict the subject's medication response using baseline assessments, and identify to which medication a patient should be assigned. This is a direction for future work, along with creating new models to assess whether predicting the placebo response on an item-level basis is stronger than predicting its cumulative power.

This provides a new trial design, one where (1) placebo responders are removed prospectively, without the need for a placebo lead-in, and (2) treatment responses are analyzed after holding constant for the likely placebo response in that individual patient-simulating a cross-over study without the second placebo phase actually being performed. Moreover, the PQRS allows estimation of the medication effect after accounting for the effect of the medication. Finally, in comparator trials, adjusting for the expected placebo response using the PQRS would allow medications to be compared directly, while subtracting out the unique placebo-related changes within that subject. These benefits—identifying placebo responders and estimating the placebo response in patients receiving a medication—are all performed without actually administering a placebo intervention. This, in turn, leads to a shortened study.

Although the total PANSS score was held constant in these models, it is possible that the placebo measure captures a higher-order feature of the 30-dimensional PANSS score such as the variance or the kurtosis of PANSS items. The effect of demographic covariates and intra-subject variation were not captured in the predictive models, a direction for future work. Not all studies here contained placebo groups, so placebo patients from other studies were used to predict the placebo response in MA groups from different studies. Because of this, cross-study variation may be blurring the true power of these methods. Despite this, the ability of this placebo model to predict treatment response in different trials validates the hypothesis that placebo responders are a unique phenotype which should be accommodated for in drug trial designs.

### REFERENCES

Faries, D. E., Heiligenstein, J. H., Tollefson, G. D., Potter, W. Z., 2001. The double-blind variable placebo lead-in period: results from two antidepressant clinical trials, Journal of clinical psychopharmacology 21, 561-568.
Fava, M., Evins, A. E., Dorer, D. J., Schoenfeld, D. A., 2003. The problem of the placebo response in clinical trials for psychiatric disorders: culprits, possible remedies, and a novel study design approach. Psychotherapy and psychosomatics 72, 115-127.
Fava, M., Mischoulon, D., losifescu, D., Witte, J., Pencina, M., Flynn, M., Harper, L., Levy, M., Rickels, K., Pollack, M., 2012. A double-blind, placebo-controlled study of aripiprazole adjunctive to antidepressant therapy among depressed outpatients with inadequate response to prior antidepressant therapy (ADAPT-A Study). Psychotherapy and psychosomatics 81, 87-97.
Howick, J., Friedemann, C., Tsakok, M., Watson, R., Tsakok, T., Thomas, J., Perera, R., Fleming, S., Heneghan, C., 2013. Are treatments more effective than placebos? A systematic review and meta-analysis. PLoS One 8, e62599.
Ivanova, A., Tamura, R. N., 2011. A two-way enriched clinical trial design: combining advantages of placebo lead-in and randomized withdrawal. Statistical methods in medical research, 0962280211431023.
Kemp, A. S., Schooler, N. R., Kalali, A. H., Alphs, L., Anand, R., Awad, G., Davidson, M., Dubé, S., Ereshefsky, L., Gharabawi, G., 2010. What is causing the reduced drug-placebo difference in recent schizophrenia clinical trials and what can be done about it? Schizophrenia bulletin 36, 504-509.
Lund, K., Vase, L., Petersen, G. L., Jensen, T. S., Finnerup, N. B., 2014. Randomised controlled trials may underestimate drug effects: balanced placebo trial design. PLoS One 9, e84104.
Rabkin, J. G., Stewart, J. W., McGrath, P. J., Markowitz, J. S., Harrison, W., Quitkin, F. M., 1987. Baseline characteristics of 10-day placebo washout responders in antidepressant trials. Psychiatry research 21, 9-22.
Rutherford, B. R., Pott, E., Tandler, J. M., Wall, M. M., Roose, S. P., Lieberman, J. A., 2014. Placebo response in antipsychotic clinical trials: a meta-analysis. JAMA psychiatry 71, 1409-1421.

### Example 2: Use of Baseline Assessments to Predict Placebo Responders and Adjust for Placebo-Quantified Response Scores

One can use the methods described herein to improve the design and/or quality of a clinical trial. FIG. 7 is a flow chart of an exemplary method 700 to improve the design and/or quality of a clinical trial. As represented in box 702, at the outset, one can perform a baseline assessment (measuring a plurality of symptoms, for example) that is used to predict placebo responders 706, placebo non-responders 704, and/or to calculate a placebo quantified response score (PQRS). Optionally, the subjects identified as predicted placebo responders are removed from the study 710. Those subjects predicted to be placebo non-responders are then randomized 708 for assignment to either the placebo group 712 or the active treatment group 714. The PQRS can then be used to adjust 716, 718 the final measures, taking into account the placebo effect for each individual subject.

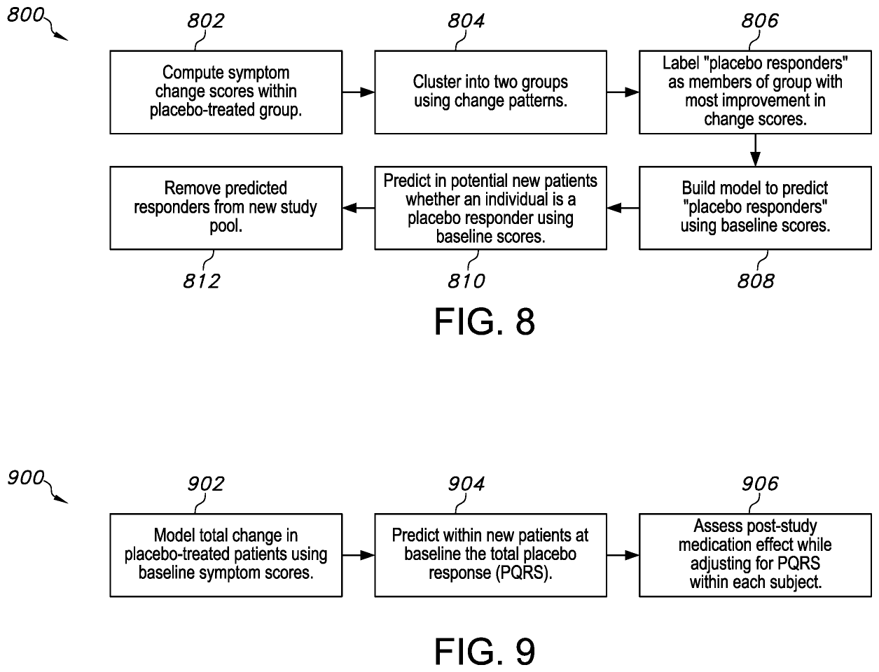

FIG. 8 is a flow chart depicting an exemplary method 800, showing steps to identify and remove placebo responders using baseline symptom scores. The method begins with computing symptom change scores within a placebo-treated group 802, and then clustering the scores into two groups using change patterns 804. Placebo responders are then labeled as members of the group with the most improvement in change scores 806, and a model is built to predict placebo responders using baseline scores 808. One can then predict in potential new patients whether an individual is a placebo responder by using baseline scores 810. Predicted placebo responders can then be removed from the new study pool 812.

FIG. 9 is a flow chart depicting an exemplary method 900, showing steps to adjust trial responses for the placebo quantified response score (PQRS). First, total change in placebo-treated patients is modeled using baseline symptom scores 902. The total placebo response (PQRS) is predicted within new patients at baseline 904. The post-study medication effect is assessed while adjusting for PQRS within each subject 906.

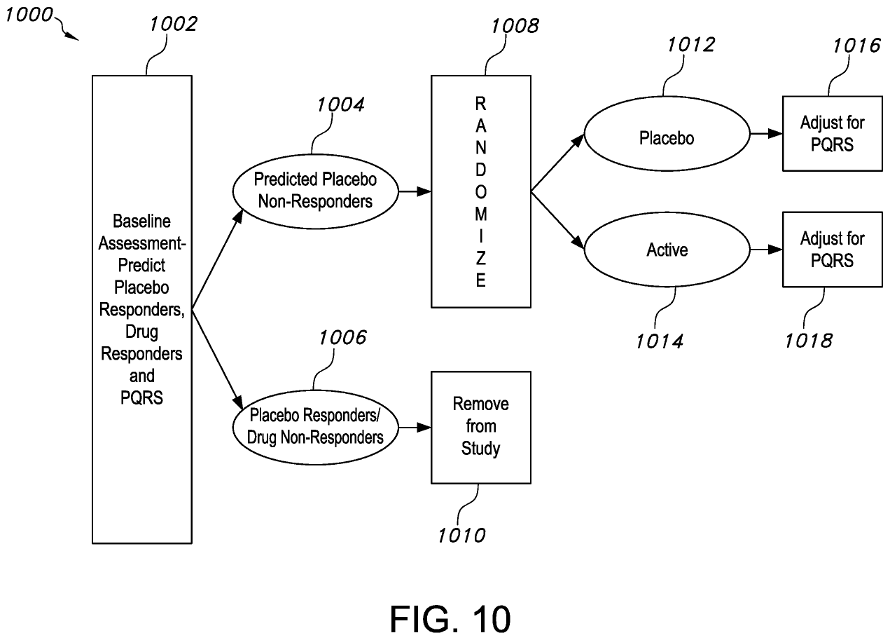

FIG. 10 is a flow chart depicting an exemplary PQRS trial design. In this method 1000, baseline assessment 1002 is used to predict placebo responders, drug responders, and PQRS. Predicted placebo non-responders are identified 1004 and randomized 1008 into placebo 1012 and active treatment 1014 groups, and outcomes can be adjusted for PQRS 1016, 1018. Placebo responders and drug non-responders are identified 1006 and removed from the study 1010.

### Example 3: Prospectively Predicting the Placebo Responders and the Placebo Risk in Major Depressive Disorder Using the Hamilton Depression Rating Scale (HAM-D 17)

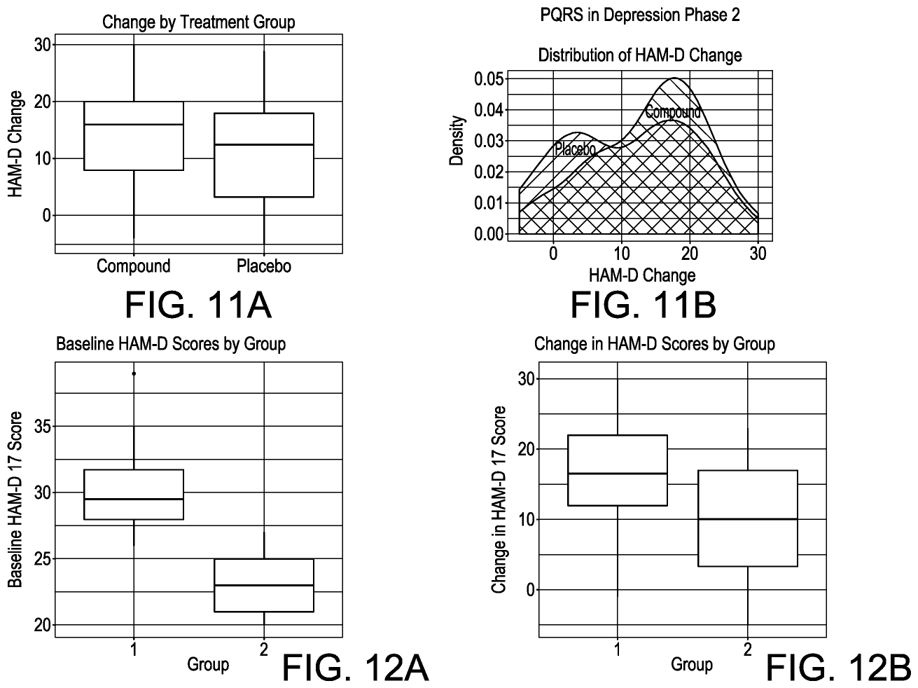

The PQRS trial design was originally developed to predict the placebo risk and placebo responders in Schizophrenia, using the Positive and Negative Syndrome Scale (PANSS) score. Here, PQRS is demonstrated for major depressive disorder (MDD), in a sample of Phase 2 trial data. The MDD dataset consists of 136 patients randomized in 1:1 to either 40 mg QD dose of a compound for 8 weeks or matching placebo, and were studied using a randomized, double-blind, parallel-group design. The inclusion criteria consisted of patients with an episode of MDD, moderate-severe severity. Patients were evaluated using the GR D-HAMD17 (Hamilton Depression Rating Scale), where a total score <=7 indicated remission at Week 8 of the study. Patients were also evaluated using the Maier-Philipp Subscale [MPS], the Clinical Global Impression-Severity (CGI-S), the Hamilton Anxiety Rating Scale (HAMA) total score, and the Patient-rated Hospital Anxiety and Depression Scale (HADS) scores, From this sample, 100 patients were used who had complete data, with 50 subjects in each treatment group. The distribution of changes scores is shown in FIG. 11B. A single question on patient pain was also included (BPIS-4).

The primary endpoint was the change in HAM-D 17 over the eight-week treatment. Because of the limited sample size the PQRS approach is demonstrated in a modified PQRS analyses, where the PQRS is predicted using all subjects instead of just the placebo group. This is similar to assuming that the placebo response in the placebo group is similar to the placebo response in the medication group. More generally, this model captures the change over the course of treatment that is common to both interventions. All PQRS values used for efficacy were predicted without that subject's data using the out-of-bag estimates, similar to a cross-validation.

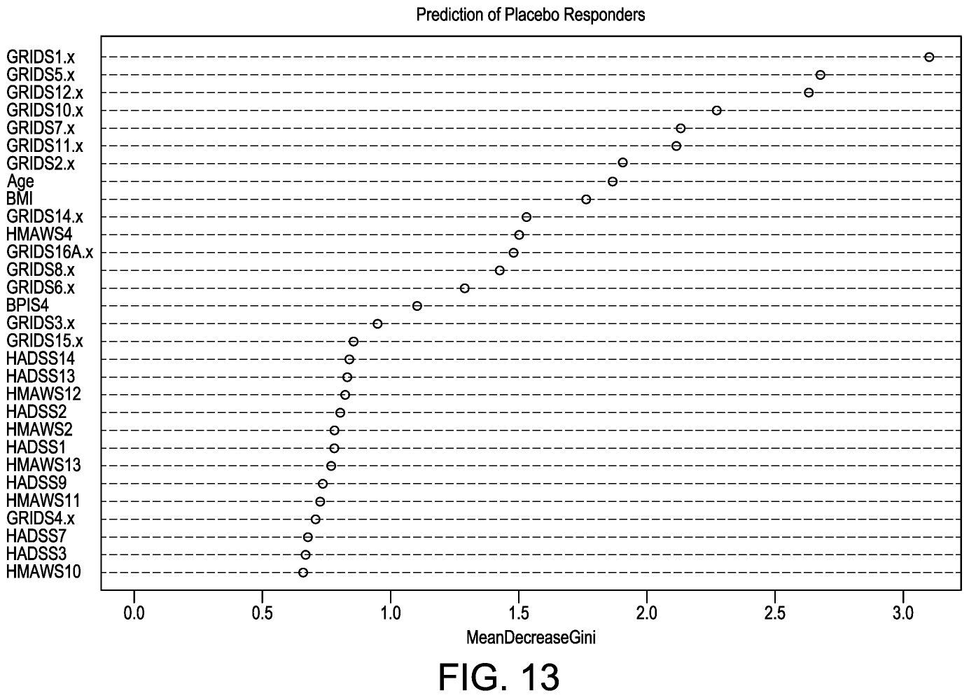

Following the PQRS procedure, spectral clustering on the change scores was used to partition the data into two groups, where group 2 is identified as the placebo non-responder group because they exhibited the largest total change. The data show that placebo responders were also more likely to have greater total baseline HAM-D scores, showing that more ill patients are more likely to be placebo responders (FIGS. 12A-12B). Of the 100 patients, 54% were predicted placebo responders. Next, a random forests machine learning model was used to predict whether someone was a placebo responder using their baseline scores. This model predicted correctly placebo responders and non-responders for 83% of subjects, where the accuracy is computed using out-of-bag classification (similar to a cross-validation). Prediction of placebo responders was more accurate than for non-responders, with corresponding error rates of 11.1% and 23.9% respectively. The most important predictors of the placebo responder groups were the HAM-ID symptoms 1, 5, 12, and 10, respectively. Although treatment group was used to predict the placebo responder status, this symptom was not important in predicting which subjects were placebo responders, after adjusting for other covariates (FIG. 13).

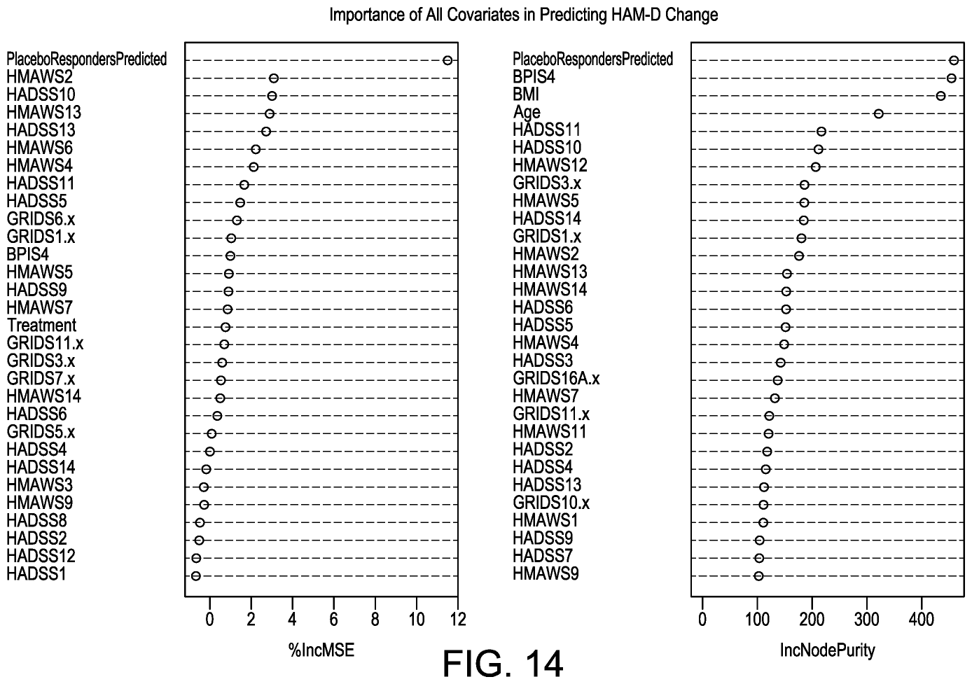

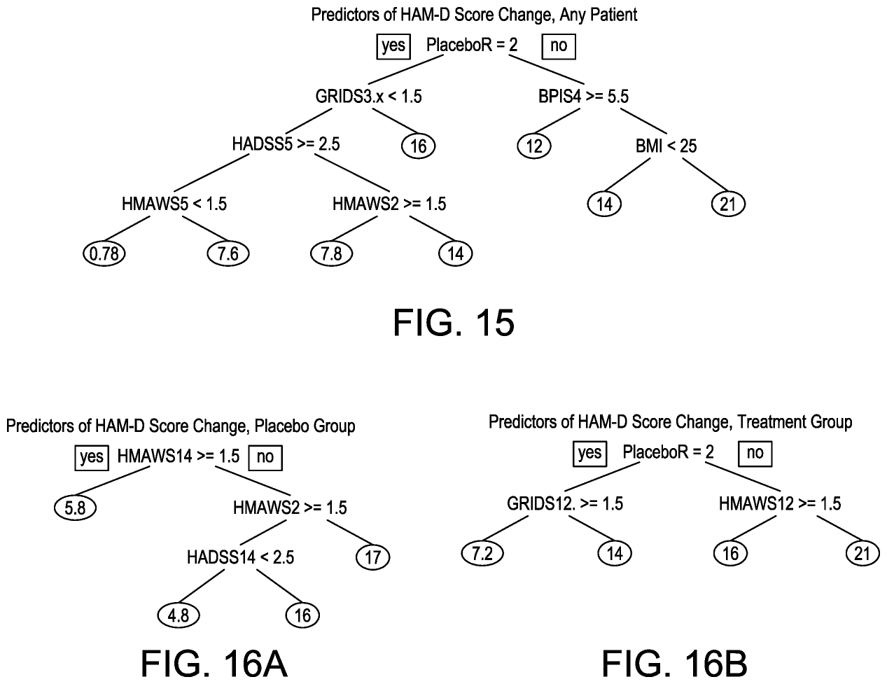

Next, the total change in HAM-D was predicted using all available covariates plus the predicted placebo responder status, where the predicted placebo responder status was determined by the random forests model (FIG. 14). A subject's predicted placebo membership was not determined by his/her change in scores, but rather by his baseline scores. The strongest predictor of change was the predicted placebo responder group membership. Other important predictors included various symptoms from all assessments used, BMI, age, and pain level (BPIS4). This is illustrated graphically in the rpart model in FIG. 15, showing a single decision tree. In FIGS. 16A-16B, the Placebo and Active treatment groups are predicted separately, to eliminate any influence of treatment on the model. The variables suggest that predicting the total treatment response is somewhat different for the active and placebo groups, and that the placebo responder subgroup is important for predicting the total treatment response in subjects receiving an active intervention.

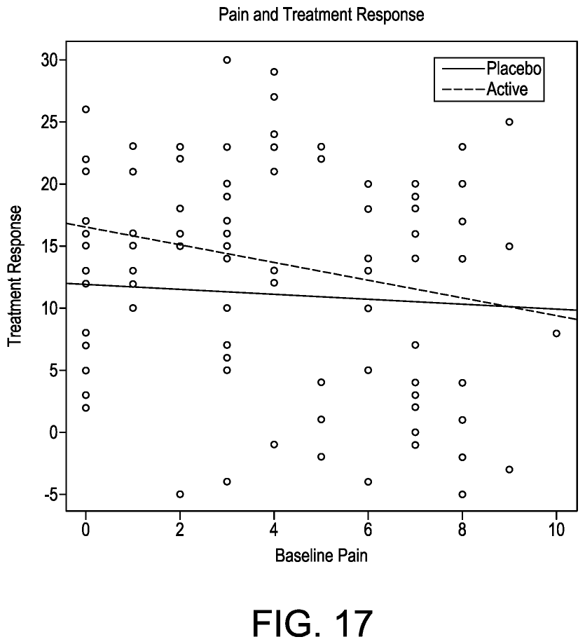

Because pain was important globally in predicting treatment response, the interaction effect between treatment assignment and baseline pain was tested, using a chi-square anova test of nested models. The interaction between these two covariates was not significant (FIG. 17), although in general increasing pain was weakly associated with diminished treatment response (p<0.10) in the baseline model which did not include medication assignment.

Next, the PQRS was predicted directly using the baseline variables. Specifically, a random forests model was used to map all baseline covariates to the total change in HAM-D score over the course of treatment. Similar to predicting the placebo responders, the model was trained over all available subjects (n=100), but the predicted values were assessed on the out-of-bag sample, similar to a cross-validation.

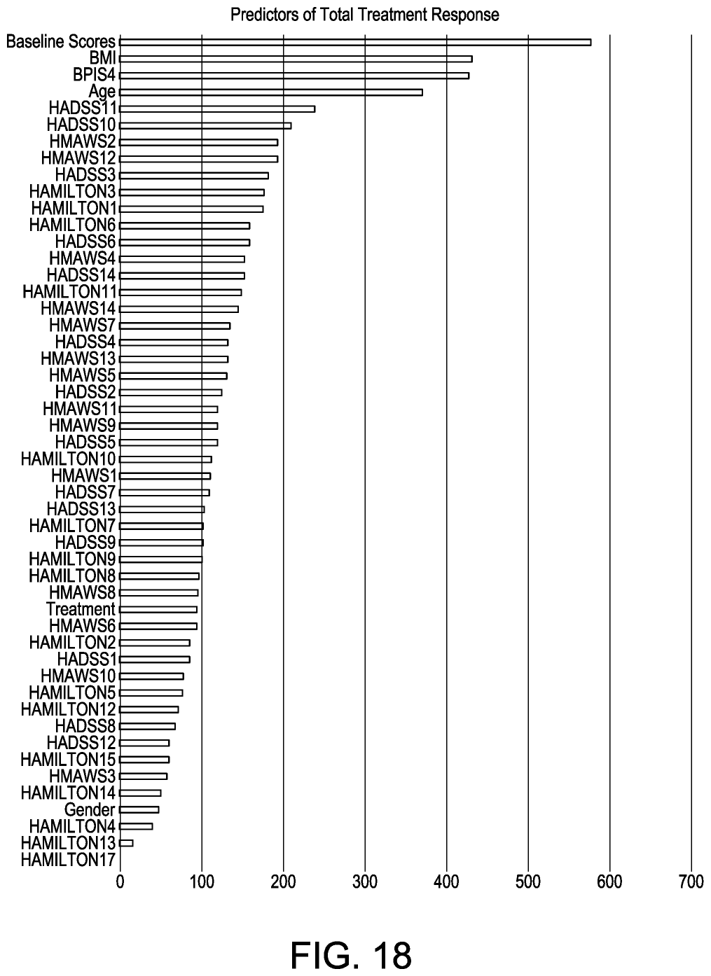

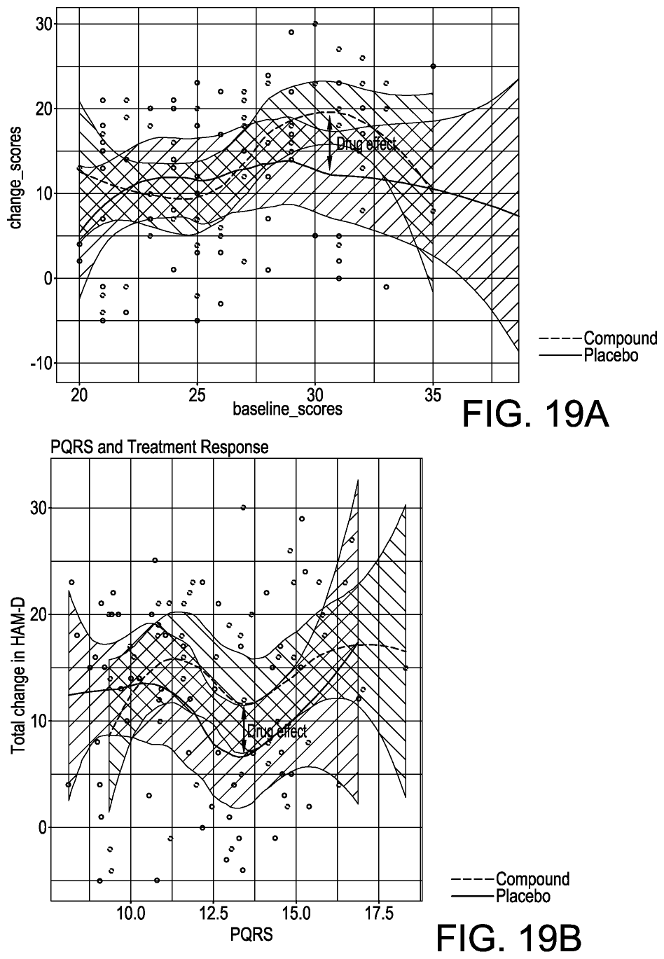

The total treatment response was then predicted with and without the PQRS in a general linear model, using the baseline scores and the intervention to predict the total change. The strongest predictors of the PQRS are shown in FIG. 18. A chi-square test of nested models was used to assess whether the PQRS helped to predict the total treatment response, above and beyond the other covariates. Table 6 shows the relationship between these covariates without the PQRS. The baseline scores significantly predicted the total treatment response, such that an increase in baseline score led to an increase in the total treatment response (p<0.05). The medication effect was not significant, although the placebo group had on average 2.7 pts smaller treatment response in HAM-D (p=0.12). When including the PQRS in Table 7, the placebo group had on average 1.01 pts reduced treatment response compared to the medication, and this effect was weakly statistically significant (p=0.056). A shown in FIGS. 19A-19B, the score changes for the Placebo and Compound group became more separable when accounting for the PQRS. The PQRS was more significant in predicting the treatment response than either the treatment or the baseline score (p<0.01). The chi-square test of nested models differed at the p=0.053 level.

In conclusion, this analysis shows that the PQRS can be used in other disorders using other instruments, and that modeling the PQRS within the placebo group or within the entire sample both lead to the PQRS benefiting statistically in predicting the total treatment response. In the MDD example with n=100 subjects, the treatment and control were nearly separable when using the PQRS, and the PQRS was highly significant (p<0.01). This suggests that the PQRS is a flexible approach for anticipating a subject's unique placebo risk, and that incorporating this information, much like age, BMI, or other relevant risk factors, can better help separate placebo interventions from active interventions.

TABLE 6
In 100 total subjects, a PQRS model compared the ability to predict a
subject's total treatment response with and without the PQRS. When
not using the PQRS, the placebo intervention was not significantly
different than the medication. Signif. codes: 0 ‘***’ 0.001
‘**’ 0.01 ‘*’ 0.05 ‘.’ 0.1 ‘’ 1

| Model 1 | Estimate | Std. Error | t value | Pr (>[t]) |
|---|---|---|---|---|
| (Intercept) | 2.0512 | 5.6348 | 0.364 | 0.7166 |
| Baseline Score | 0.4494 | 0.2064 | 2.177 | 0.0319* |
| Treatment: Placebo | −2.6895 | 1.6992 | −1.583 | 0.1167 |

TABLE 7
When including the PQRS, the medication was weakly superior to the
medication in 100 total subjects, with 50 patients in each treatment
group (p = 0.056). The PQRS was more significant in predicting
the treatment response than either the treatment assignment or the
baseline score (p < 0.01). Signif. codes: 0 ‘***’ 0.001 ‘**’
0.01 ‘*’ 0.05 ‘.’ 0.1 ‘’ 1

| Model 2 | Estimate | Std. Error | t value | Pr (>[t]) |
|---|---|---|---|---|
| (Intercept) | 6.7153 | 6.0567 | 1.109 | 0.27032 |
| PQRS | 0.774 | 0.2637 | 2.935 | 0.00418** |
| Baseline Score | −3.6405 | 1.7462 | −2.085 | 0.03974* |
| Treatment: Placebo | −1.0109 | 0.5223 | −1.935 | 0.05587. |

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of producing a placebo responder profile from a treatment group of subjects, the method comprising:
(a) measuring a plurality of symptoms in a first population of subjects receiving a placebo treatment before and after said treatment to obtain a plurality of baseline symptom scores and post-treatment symptom scores;
(a) generating a plurality of placebo difference scores, wherein a placebo difference score is the difference between the post-treatment symptom score of a subject and the baseline symptom score of the same subject;
(b) performing a clustering analysis to create two distinct profiles of placebo difference scores generated in (b), wherein a profile associated with placebo responders exhibits greater total difference scores than a profile associated with non-responders; and
(c) analyzing the baseline symptom scores of the placebo responders identified in (c) to create a baseline placebo responder profile.

2. The method of claim 1, wherein the clustering analysis is a spectral clustering analysis.

3. The method of claim 1, further comprising:
(e) measuring the plurality of symptoms in a second population of subjects receiving a psychopharmacologic treatment before said psychopharmacologic treatment to obtain a plurality of baseline symptom scores; and
(f) identifying subjects in the second population whose baseline symptom scores exhibit the baseline placebo responder profile of (d) as placebo responders.

4. A method of obtaining a placebo quantified response score (PQRS) from a treatment group of subjects, the method comprising:
(a) measuring a plurality of symptoms in a first population of subjects receiving a placebo treatment before and after said treatment to obtain a plurality of baseline symptom scores and post-treatment symptom scores;
(b) generating a plurality of placebo difference scores, wherein a placebo difference score is the difference between the post-treatment symptom score of a subject and the baseline symptom score of the same subject;
(c) modeling a total post-treatment symptom score measured after treatment with placebo as a function of baseline symptom scores; and
(d) generating a PQRS using the modeling of step (c) to predict a post-treatment symptom score attributable to placebo based on a baseline symptom score.

5. The method of claim 4, further comprising:
(e) measuring the plurality of symptoms in a second population of subjects receiving a psychopharmacologic treatment before and after said psychopharmacologic treatment to obtain a plurality of baseline symptom scores and post-treatment symptom scores;
(f) generating a PQRS for each subject in the second population; and
(g) adjusting the post-treatment scores measured in the second population of subjects using the PQRS for that subject.

6. A method of improving the quality and efficiency of a clinical trial, the method comprising:
(a) obtaining measured responses of a plurality of symptoms in a population of subjects prior to a trial treatment;
(b) analyzing the measured responses obtained in (a) to generate a placebo quantified response score (PQRS);
(c) measuring the subjects' responses to treatment with either a trial medication or a placebo; and
(d) adjusting the measured responses obtained in (c) to account for the PQRS.

7. The method of claim 6, wherein the adjusting of (d) comprises removing measured responses of subjects whose PQRS identifies them as placebo responders from the responses measured in step (c), and/or subtracting the PQRS from the measured response of each subject.

8. The method of claim 6, wherein steps (c) and (d) are performed only for those subjects whose PQRS identifies them as placebo non-responders.

9. The methods of any of the preceding claims, wherein the plurality of symptoms is the set of symptoms of the Positive and Negative Symptom Scale (PANSS), Hamilton Anxiety Rating Scale (HAM-A), or the Hamilton Depression Rating Scale (HAM-D).

10. The method of claim 7, wherein the PQRS identifies a subject as a placebo responder if the PQRS is higher than a threshold identified using the methods described herein to distinguish placebo responders from placebo non-responders.

11. The method of claim 4, wherein the trial medication is a psychopharmacologic treatment.

12. The method of claim 1, wherein the psychopharmacologic treatment or trial medication comprises one or more medications selected from the group consisting of Olanzapine, Paliperidone, Paliperidone Palmitate, Quetiapine, and Risperidone.

13. A method of performing clinical trial of a treatment, the method comprising:
performing the method of claim 6, wherein the measuring of step (c) comprises measuring the subjects' responses to treatment with a trial medication; and
wherein the clinical trial does not require administration of a placebo.

14. The method of claim 13, wherein the treatment is a psychopharmacologic treatment.

15. A method of adjusting dosing of active medication within a clinical trial, the method comprising:
(a) generating a PQRS for a subject according to the method of claim 4; and
(b) assigning a lower dose of active medication if the subject has a high PQRS.

16. The method of claim 1, wherein the measured responses comprise functional magnetic resonance imaging (fMRI) of the brain of the subjects.

17. A system for performing a clinical trial, the system comprising:
(a) a computer readable memory storing data describing measured responses of a plurality of symptoms in a population of subjects prior to a trial treatment;
(b) an analyzer that generates a placebo quantified response score (PQRS) from measured responses stored as the data of (a);
(c) an analyzer that receives the subjects' measured responses to treatment with a trial medication; and
(d) a processor that adjusts the measured responses obtained in (c) to account for the PQRS.

18. A system for performing a clinical trial, the system comprising:
means for performing each of steps (a) to (d) of claim 13
wherein the clinical trial does not require administration of a placebo.

19. A computer system for analyzing data resulting from a clinical trial, the system comprising:
(a) a first processor that analyzes measured responses of a plurality of symptoms in a population of subjects prior to a trial treatment to generate a placebo quantified response score (PQRS);
(b) a second processor that analyzes the subjects' measured responses to treatment with a trial medication; and
(c) a third processor that adjusts the measured responses obtained in (b) to account for the PQRS,
wherein the first, second, and third processors are the same processor, separate processors, or a combination thereof.

20. A computer readable non-transitory storage medium storing software for analyzing clinical data, the software comprising:
(a) software that analyzes measured responses of a plurality of symptoms in a population of subjects prior to a trial treatment to generate a placebo quantified response score (PQRS);
(b) software that analyzes the subjects' measured responses to treatment with a trial medication; and
(c) software that adjusts the measured responses obtained in (b) to account for the PQRS.
